(19) United States
(12) Patent Application Publication
(10) Pub. No.: US 2013/0190317 A1
(43) Pub. Date: Jul. 25, 2013

(54) COMBINATION OF COMPOUNDS FOR TREATING OR PREVENTING SKIN DISEASES

(71) Applicants: Jean-Paul Chappuis, Valbonne (FR); Emmanuelle At, Antibes (FR)

(72) Inventors: Jean-Paul Chappuis, Valbonne (FR); Emmanuelle At, Antibes (FR)

(73) Assignee: GALDERMA RESEARCH & DEVELOPMENT SNC, Biot (FR)

(21) Appl. No.: 13/821,155

(22) PCT Filed: Aug. 5, 2011
(86) PCT No.: PCT/EP2011/063528

§ 371 Date: Apr. 11, 2013

Related U.S. Application Data
(60) Provisional application No. 61/344,499, filed on Aug. 6, 2010.

(51) Int. Cl. A61K 31/498; A61K 31/194

(52) U.S. Cl. CPC A61K 31/498; A61K 31/194
USPC 514/249; 514/574

(57) ABSTRACT

The invention relates to a combination of compounds for treating skin diseases and particularly rosacea and ocular rosacea. It is the combination of a compound selected from azelaic acid and salts thereof with a compound of the alpha-1 or alpha-2 adrenergic receptor agonist family. The invention also relates to a product in the form of a kit containing: (a) a first composition comprising a compound selected from azelaic acid and salts thereof, and (b) a second composition different from the first one and comprising a compound of the alpha-1 or alpha-2 adrenergic receptor agonist family, as a combination product for application thereof as a medicament for treating and/or preventing skin diseases and in particular rosacea and ocular rosacea, wherein said first and second compositions can be applied simultaneously, separately or with a time delay.

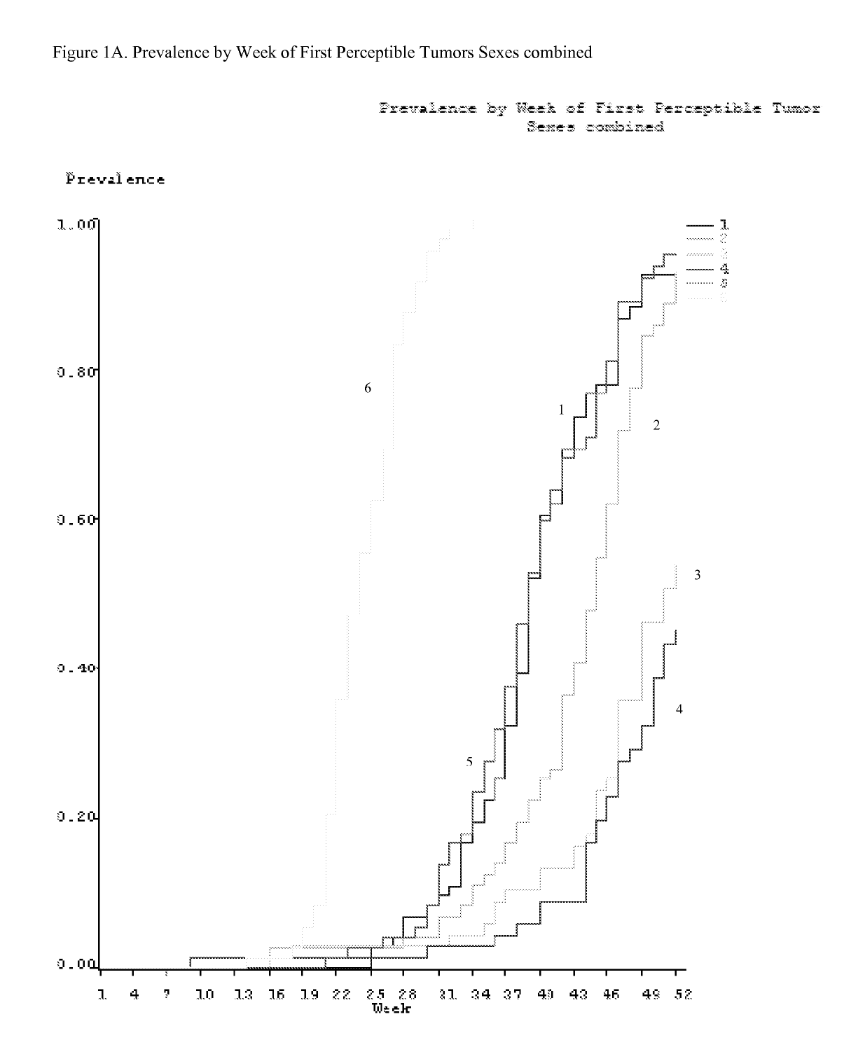

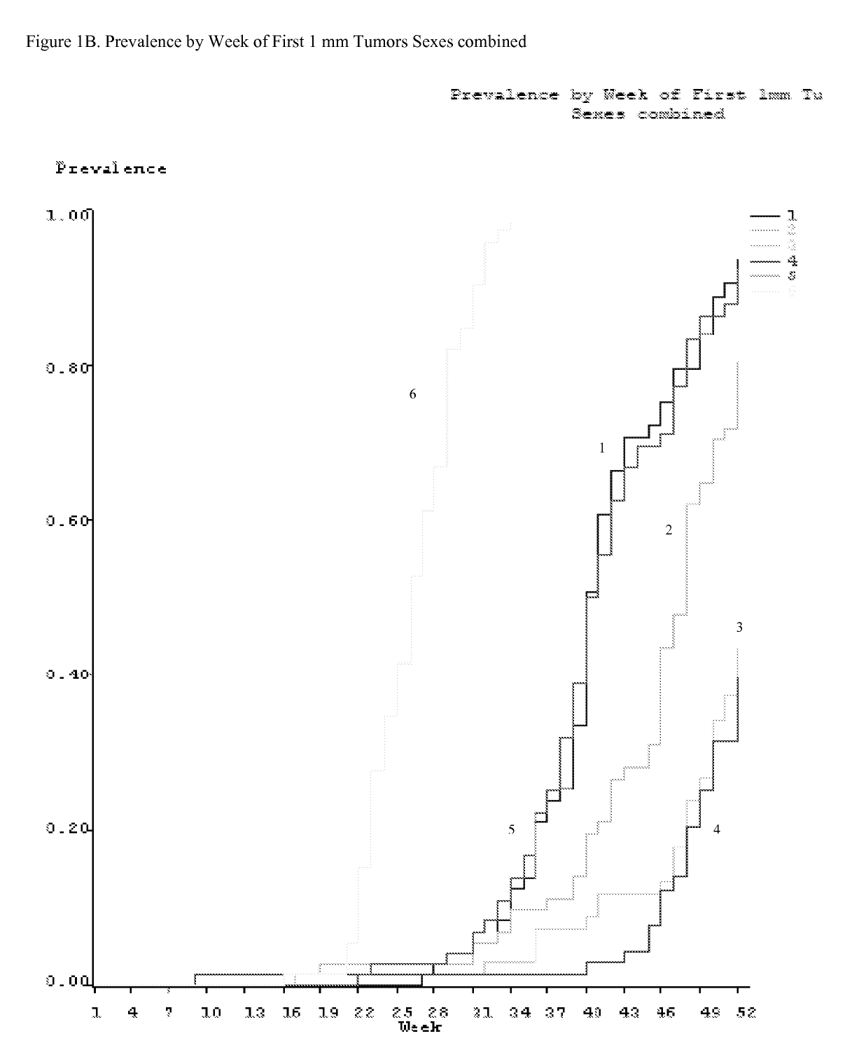

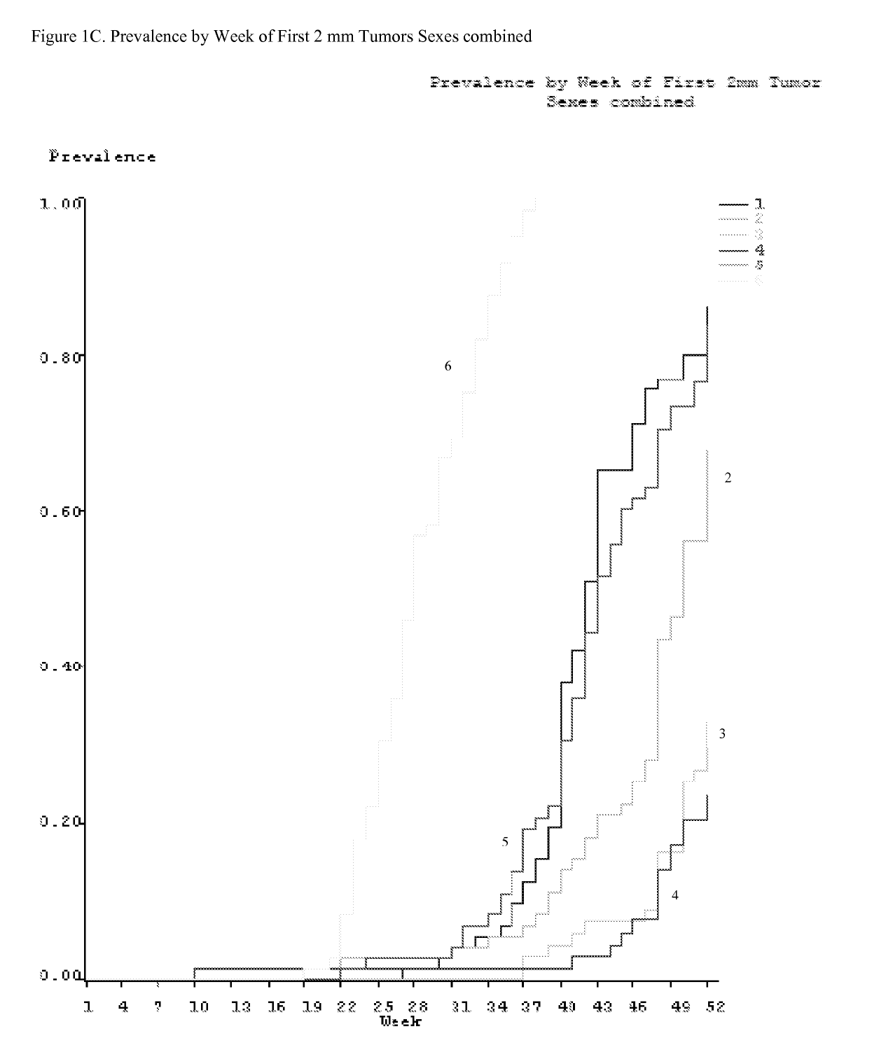

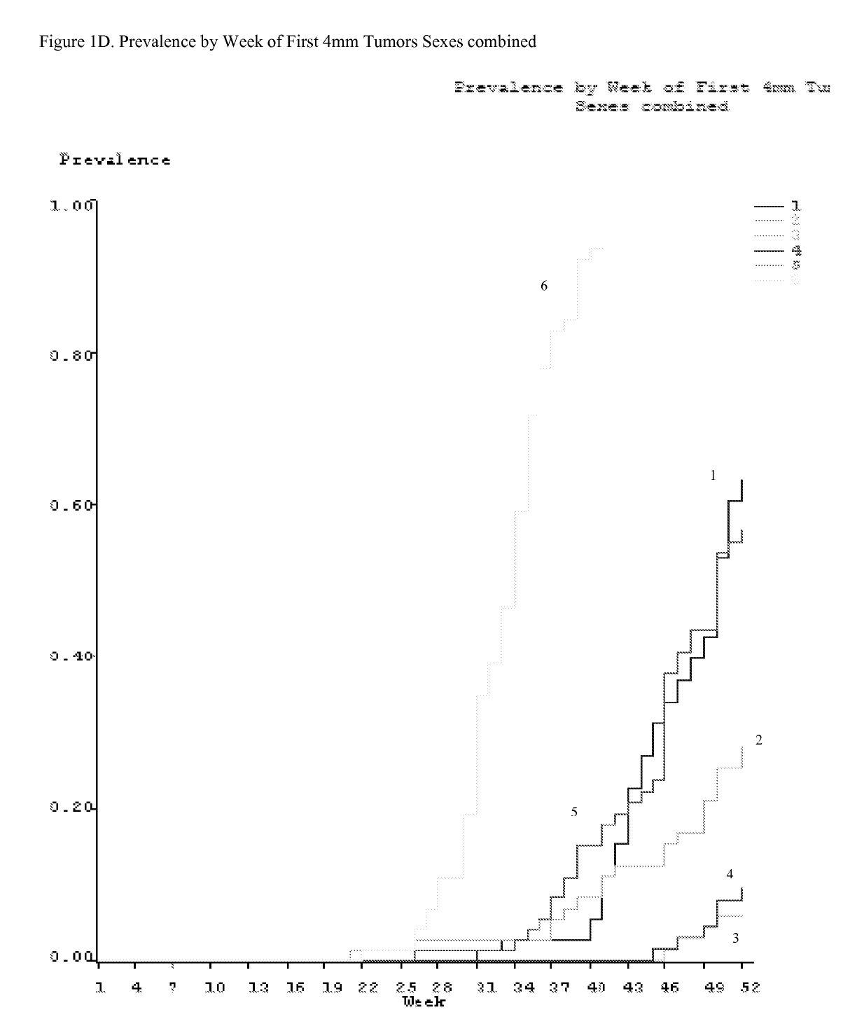

The invention relates to a combination of compounds for treating skin diseases in humans, particularly rosacea and ocular rosacea.

Rosacea is a common chronic and progressive inflammatory dermatitis related to vascular relaxation. It mainly affects the central part of the face and is characterized by a reddening of the face or hot flushes, facial erythema, papules, pustules, telangiectasia and sometimes ocular lesions called ocular rosacea. In serious cases, particularly in men, the soft tissue of the nose can swell and produce a bulbous swelling called rhinophyma.

Rosacea generally occurs between the ages of 25 and 70, and it is much more common in people with a fair complexion. It affects more particularly women, although this disease is generally more severe in men. Rosacea is chronic and persist for years with periods of exacerbation and remission.

The pathogenesis of rosacea is poorly understood. Many factors may be involved without necessarily inducing this disease. They are, for example, psychological factors, gastrointestinal disorders, environmental factors (exposure to the sun, temperature, humidity), emotional factors (stress), food-related factors (alcohol, spices), hormonal factors, vascular factors, or even an infection with Helicobacter pylori.

Conventionally, rosacea is treated orally or topically with antibiotics such as tetracyclines, erythromycin, or clindamycin, but also with vitamin A, salicylic acid, antifungal agents, steroids or metronidazole (an antibacterial agent) or with isotretinoin in severe forms or else with anti-infectives such as benzoyl peroxide.

The treatment of rosacea with ivermectin, which targets the Demodex folliculorum parasite present on the skin of patients, is also known (U.S. Pat. No. 5,952,372).

Azelaic acid (or 1,7-heptanedicarboxylic acid) is known in the prior art for its anti-acne and keratolytic properties. Azelaic acid has an antibacterial activity on P. acnes and on S. epidermidis. It inhibits keratinocyte proliferation, reduces the level of free fatty acids in sebaceous secretions and also has an anti-inflammatory activity.

Patent application WO 2004/022046 describes a method for treating rosacea by topical application of a composition based on azelaic acid and on metronidazole.

The treatment of rosacea with alpha-1 or alpha-2 adrenergic receptor agonists is also known (U.S. 2006/0171974A1, U.S. 2005/0165079A1, U.S. 2005/0020600A1).

These treatments have side effects that are unpleasant for the patient, such as irritation or intolerance phenomena. Furthermore, none of the existing treatments make it possible to effectively treat and/or prevent all the symptoms associated with rosacea.

Taking into account the aforesaid, there is therefore a need to produce a more effective treatment for rosacea, which does not have the side effects observed in the prior art. There is in particular a need to produce a composition which confers a greater tolerance of the active ingredients, while at the same time reducing their side effects.

Brimonidine is, for its part, used in ophthalmology for decreasing intraocular pressure in individuals suffering from open-angle glaucoma or from intraocular hypertension (elevated pressure inside the eye).

Surprisingly, the applicant has observed that a combination of azelaic acid with a compound of the alpha-1 or alpha-2 adrenergic receptor agonist family allows a more effective treatment of rosacea, with fewer side effects irrespective of the duration of application of this combination. In particular, such a combination makes it possible to substantially reduce the duration of treatment and to obtain a greater reduction in the symptoms of rosacea. This combination may also make it possible to eliminate the rebound effect normally observed at the end of treatment with alpha-1 or alpha-2 adrenergic receptor agonists.

A subject of the invention is a combination of a compound selected from azelaic acid and salts thereof with a compound of the alpha-1 or alpha-2 adrenergic receptor agonist family, for application thereof as a medicament for treating and/or preventing skin diseases and particularly rosacea and ocular rosacea.

A subject of the invention is also the use of a combination of a compound selected from azelaic acid and salts thereof with a compound of the alpha-1 or alpha-2 adrenergic receptor agonist family for the production of a medicament intended for treating and/or preventing skin diseases and particularly rosacea and ocular rosacea.

In a preferred embodiment, the composition according to the invention is intended for the treatment of facial rosacea.

A subject of the present invention is also a pharmaceutical, in particular dermatological, composition comprising, in a physiologically acceptable medium, at least one compound selected from azelaic acid and salts thereof and at least one compound of the alpha-1 or alpha-2 adrenergic receptor agonist family, intended for treating and/or preventing skin diseases and particularly rosacea and ocular rosacea.

A subject of the invention is preferentially a pharmaceutical, in particular dermatological, composition comprising, in a physiologically acceptable medium, at least brimonidine and azelaic acid.

The term “dermatological composition” is intended to mean a pharmaceutical composition applied to the skin.

The term “physiologically acceptable medium” is intended to mean a medium that is compatible with the skin, the mucous membranes and/or the skin appendages.

The term “skin diseases” is intended to mean cutaneous and ocular disorders. By way of nonlimiting example, mention may be made of acne, hyperseborrhoea, facial rosacea, ocular rosacea, psoriasis and atopic dermatitis.

The skin disease is more particularly facial rosacea or ocular rosacea.

A subject of the invention is also the use of such a composition for producing a medicament intended for preventing and/or treating skin diseases and particularly rosacea and ocular rosacea.

A subject of the invention is also a product in the form of a kit containing:

(a) a first composition comprising a compound selected from azelaic acid and salts thereof, and

(b) a second composition different from the first one and comprising a compound of the alpha-1 or alpha-2 adrenergic receptor agonist family,

as a combination product for application thereof as a medicament for treating and/or preventing skin diseases and particularly rosacea and ocular rosacea, wherein said first and second compositions can be applied simultaneously, separately or with a time delay.

A subject of the invention is also the use of a product in the form of a kit containing:

(a) a first composition comprising azelaic acid and, and

(b) a second composition different from the first one and comprising a compound of the alpha-1 or alpha-2 adrenergic receptor agonist family,

as a combination product for producing a medicament intended for treating and/or preventing skin diseases and particularly rosacea and ocular rosacea, wherein said first and second compositions can be applied simultaneously, separately or with a time delay.

The azelaic acid according to the invention can be used as it is, or else in the form of a salt with a pharmaceutically acceptable base.

According to the invention, the compound of the alpha-1 adrenergic receptor agonist family is advantageously selected from metaraminol bitartrate, midodrine, methoxamine, mephentermine, phenylephrine, oxymetazoline, tetrahydrozoline, naphazoline or xylometazoline, or their salts.

More particularly, the compound of the alpha-1 adrenergic receptor agonist family as defined above is in hydrochloride or bitartrate form.

In a preferred embodiment, the compound of the alpha-1 adrenergic receptor agonist family is oxymetazoline.

According to the invention, the compound of the alpha-2 adrenergic receptor agonist family is advantageously chosen from apraclonidine, brimonidine, clonidine, mirtazapine, dexmedetomidine, guanbenz acetate, lidamidine, lofexidine, methyldopa, rilmenidine, talipexole, tiamenidine, tizanidine, tolonidine or their salts.

More particularly, the compound of the alpha-2 adrenergic receptor agonist family as defined above is in tartrate form.

More particularly, the compound of the alpha-2 adrenergic receptor agonist family may be brimonidine or its tartaric salt.

The combination according to the invention contains more particularly azelaic acid and a compound of the alpha-2 adrenergic receptor agonist family.

Preferentially, the combination according to the invention contains brimonidine and azelaic acid.

In another preferred embodiment, the combination according to the invention contains oxymzetazoline and metronidazole.

In the context of the present invention, a combination of a compound selected from azelaic acid and salts thereof with a compound of the alpha-1 or alpha-2 adrenergic receptor agonist family means that said combined compounds can be either present in the same composition, or present separately from one another in separate compositions, forming for example a product in the form of a kit. In other words, these compounds are intended to be administered to a patient as part of a single treatment, i.e. over a common period of treatment, either at the same time, optionally being included in one and the same composition, or at different moments. Furthermore, they can be administered by identical or different administration methods and/or be included in identical or different compositions.

The combination of the abovementioned compounds present separately in separate compositions, and in particular in the case of a product in the form of a kit, makes it possible to limit the interactions of the azelaic acid with the compound(s) of the alpha-1 or alpha-2 adrenergic receptor agonist family. This also makes it possible to limit as much as possible the interaction of the azelaic acid with the numerous excipients normally contained in a single composition, and in particular the excipients contained in the composition comprising the compounds of the alpha-1 or alpha-2 adrenergic receptor agonist family. The compositions according to the invention, applied simultaneously or successively, are thus very well tolerated, precise in terms of amount of active compounds delivered, and practical to use. They also offer the patients comfort and hydration.

In the case of a combination of the abovementioned compounds present separately in separate compositions, and in particular in the case of a product in the form of a kit, the azelaic acid can first be applied to the skin of a patient, and then a compound of the alpha-1 or alpha-2 adrenergic receptor agonist family can be applied, or vice versa.

In the compositions according to the invention, the azelaic acid is present at a concentration of between 0.01 and 40% by weight, relative to the total weight of the composition comprising it, preferably between 1 and 20% by weight. When a composition comprises several of these compounds, their total concentration is included in the abovementioned amounts.

In the compositions according to the invention, the compound of the alpha-1 or alpha-2 adrenergic receptor agonist family is present at a concentration of between 0.01 and 20% by weight, relative to the total weight of the composition, preferably between 0.02 and 10%, particularly preferably between 0.05 and 5% by weight, relative to the total weight of the composition. When a composition comprises several of these compounds, their total concentration is included in the abovementioned amounts.

Particularly preferably, the combination comprises a compound selected from azelaic acid and salts thereof, present at a concentration of between 1 and 20% by weight, relative to the total weight of the composition comprising it, and a compound of the alpha-1 or alpha-2 adrenergic receptor agonist family present at a concentration of between 0.01 and 5% by weight, relative to the total weight of the composition.

Said composition according to the invention contains more particularly azelaic acid and a compound of the alpha-2 adrenergic receptor agonist family.

Preferentially, the composition according to the invention comprises brimonidine and azelaic acid.

The combination according to the invention and the compositions comprising the compounds of this combination are in particular intended for topical application to the skin and/or for ocular application to the eyes.

The compositions of the invention also comprise a pharmaceutically or cosmetically acceptable vehicle, i.e. a vehicle suitable for use in contact with human cells, without toxicity, irritation, undue allergic response and the like, and proportioned at a reasonable advantage/risk ratio.

The compositions of the invention may also comprise at least one other therapeutic agent capable of increasing the efficacy of the treatment.

The compositions of the invention may also comprise any additive normally used in the pharmaceutical or dermatological field, which is compatible with azelaic acid and/or the compound of the alpha-1 or alpha-2 adrenergic receptor agonist family that is/are present.

Mention may in particular be made of sequestering agents, antioxidants, sunscreens, preservatives, for example DL-alpha-tocopherol, fillers, electrolytes, humectants, colourants, customary inorganic or organic bases or acids, fragrances, essential oils, cosmetic active agents, moisturizers, vitamins, essential fatty acids, sphingolipids, artificial tanning compounds such as DHA, agents for soothing and protecting the skin, such as allantoin, propenetrating agents, gelling agents or a mixture thereof. Of course, those skilled in the art will take care to select this or these optional additional compound(s), and/or the amount thereof, in such a way that the advantageous properties of the composition according to the invention are not, or not substantially, impaired.

The administration may be carried out topically, enterally or orally, parenterally or ocularly.

Among these routes of administration, the topical route and the ocular route are particularly preferred.

The compositions of the present invention may be in any of the galenical forms normally used for topical application, in particular in the form of solutions, lotions, gels, emulsions of liquid or semi-liquid consistency of the milk type, obtained by dispersion of a fatty phase in an aqueous phase (O/W) or vice versa (W/O), or suspensions or emulsions of soft, semi-liquid or solid consistency, of the cream or ointment type, or else microemulsions, microcapsules, microparticles or vesicular dispersions of ionic and/or nonionic type.

In a preferred embodiment, the composition is a cream, a solution or a gel.

In a preferred embodiment, the composition according to the invention comprises at least one ingredient or a combination of several ingredients as described below in the following pages.

In one embodiment, the composition according to the invention may comprise at least one gelling agent.

By way of example of a gelling agent, mention will preferably be made of: a Carbomer such as Carbopol 980 NF, Carbopol ETD2020, Carbopol 974P NF, Carbopol Ultrez-20® sold by Noveon;

a cellulose derivative such as hydroxypropylmethylcellulose sold under the name Methocel E4M® by Dow or hydoxyethylcellulose sold under the name Natrosol 250HHX® by IMCD;

a Polysaccharide such as xanthan gums sold under the name Satiaxane UCX911® by IMCD or Xantural 180® by Kelco, guar gum sold under the name N-Hance HP 40® by IMCD;

a Polyacrylamide such as polyacrylamide/C13-14 isoparaffin/laureth-7 sold under the name Sepigel® 305 by Seppic, the mixture acrylamide, acrylamido-2-methylpropanesulfonic acid (AMPS) copolymer dispersion 40%/isohexadecane sold under the name Simulgel® 600PHA by Seppic,

or a Carraghenan such as lambda or iota carraghenans sold under the name Viscarin® GP 209 or Gelcarin® PC379 by IMCD or a mixture.

In a preferred embodiment, the gelling agent is Carbopol 974P NF, Carbopol 980, or Simulgel 600PHA.

The gelling agents may be used singly or in combination of two or more. They are preferably incorporated in amounts from 0.1 to 30% by weight, particularly, from 0.1 to 10% and more preferably from 0.2 to 5% by weight (hereinafter may referred to simply as %) based on the total weight of the composition.

In one embodiment, the composition according to the invention may comprise surfactant-emulsifiers. In one preferred embodiment, the surfactant-emulsifier is a non-ionic surfactant-emulsifier.

Among these compounds, mention may be made, by way of examples, of the Glyceryl stearate and PEG 100 stearate sold under the name Arlacel® 165Flakes, PEG-6 and PEG 32 stearate sold under the name Tefose® 1500, PEG 20 methyl glucose sesquistearate sold under the name Glucamate® SSE 20, POE (21) stearyl ether sold under the name Brij 721®, ceteareth-20 sold under the name Eumulgin B2®, methyl glucose sesquistearate sold under the name Glucate0 SS, sorbitan monostearate sold under the name Span® 60 by Croda, or Sucroses esters such as Sucrose laurate, sucrose stearate, sucrose dilaurate and sucrose tristearate sold respectively under the name Surfhope® C1216, Surfhope® C1811, Surfhope® C1205, Surfhope0 C1803 by Mitsubishi Kagaku Foods Corporation.

In a preferred embodiment, the surfactant-emulsifier is Tefose 1500.

The composition according to the invention advantageously comprises up to 15% by weight of suitable surfactant-emulsifier, preferably from 2 to 15% by weight, relative to the total weight of the composition.

In one embodiment, the composition according to the invention may comprise a dispersing agent:

Among these compounds, mention may be made, by way of examples, of the Polysorbate 80 sold under the name Tween® 80, Phosphatidylcholine sold under the name Phospholipon® 90G by Phospholipid GmbH, Phospholipids sold under the name Phopholipon® 80 by Phospholipid GmbH.

In a preferred embodiment, the dispersing agent is Polysorbate 80, Phosphatidylcholine.

The composition according to the invention advantageously comprises preferably from 5 and 15% by weight of suitable dispersing agent, relative to the total weight of the composition.

In one embodiment, the composition according to the invention may comprise an oily phase. Preferably, the oily phase may comprise one or more oil.

Among these compounds, mention may be made, by way of examples, of the vegetable oil such as almond oil; Animal oil such as perhydrosqualene; Synthetic oil such as isopropyl palmitate sold under the name Crodamol® IPP by Croda, isopropyl myristate sold under the name Crodamol® IPM by Croda, caprylic/capric triglycerides sold under the name Miglyol 812N ® by Univar; Silicone oil such as dimethicone sold under the name Q7-9120 Silicone fluid®, cyclomethicone sold under the name ST-Cyclomethicone 5NF®; Mineral oil: such as paraffin oil sold under the name Primol® 352, Marcol® 152 by Esso. In a preferred embodiment, the oil is Miglyol 812N.

The oil of the composition according to the invention may be present at a content of between 2 and 10% by weight relative to the total weight of the composition.

In one embodiment, the oily phase of the composition according to the invention may comprise also a wax, fatty acids, or fatty alcohols or a mixture ranging in total from 2 to 15% by weight relative to the total weight of the composition.

Among these compounds, mention may be made, by way of examples, of the Stearic acid, Cetyl alcohol sold under the name Speziol® C18 by Cognis, stearyl alcohol sold under the name Speziol® C16 by Cognis, cetostearyl alcohol sold under the name Speziol® C16-18 by Cognis, Glyceryl dibehenate (and) tribehenin (and) glyceryl behenate sold under the name Compritol® 888 by Gattefosse or glyceryl stearate sold under the name Geleol®.

In a preferred embodiment, the composition may comprise Speziol C18 or Speziol C16-18.

In one embodiment, the composition according to the invention comprises at least one solvent ranging in total from 50 to 80% by weight relative to the total weight of the composition. Among these compounds, mention may be made, by way of examples, of the Purified water; Glycols such as propylene glycol, dipropylene glycol, propylene glycol dipelargonate, lauroglycol; Diethylene glycol mono ethyl ether sold under the name Transcutol® HP; Alcohols such as ethanol, isopropanol, butanol.

In a preferred embodiment, the composition may comprise Purified water, Propylene glycol or Transcutol HP.

The solvents may be used singly or in combination of two or more.

In an alternative embodiment, the composition according to the invention may comprise some additives ranging in total from 10 to 25% by weight relative to the total weight of the composition.:
Preservatives: methyl paraben (sold under the name Nipagin® M), propyl paraben (sold under the name Nipasol® M), sorbic acid, phenoxyethanol, benzalkonium chloride, gluconolactone, potassium sorbate, benzylic alcohol;
Antioxydants: butylhydroxyanisole (BHT), butylhydroxyanisole (BHA), α-tocopherol, ascorbic acid;
Emollients: glycerin, sorbitol, polyethylene glycol (sold under the name Lutrol® E400), amino acids;
Vitamins: Vitamin B3 (Niacinamide);
pH regulators: sodium hydroxide, triethanolamine;
Chelating agents: diethylene triamine pentaacetic acid (DPTA), ethylene diamine tetra-acetic (EDTA) sold in the name Titriplex® III;
Charges: Titanium dioxide sold in the name Unipure® White LC987.

They are preferably incorporated each in amounts from 0.01 to 15% by weight based on the total weight of the composition.

In one preferred embodiment, the composition according to the invention comprises:

at least one surfactant-emulsifier,

At least one solvent,

At least one gelling agent,

And at least one dispersing agent.

In another preferred embodiment, the composition according to the invention comprises:

At least one solvent,

at least one gelling agent,

at least one dispersing agent.

and at least one additive.

In another preferred embodiment, the composition according to the invention comprises:

at least one surfactant-emulsifier,

At least one solvent,

at least one gelling agent,

at least one oily phase,

at least one dispersing agent.

and at least one additive.

By way of illustration and without being in any way limiting in nature, various formulations of compositions according to the invention will now be given.

### EXAMPLE 1

### Brimonidine 0.20%/Acide Azelaique 15% Solution

| | % by weight relative |
|---|---|
| | to the total weight |
| Ingredients | of the composition |
| Azelaic acid | 15.00 |
| Brimonidine tartrate | 0.20 |
| Ethylene diamine tetra-acetic acid (EDTA) | 0.1 |
| Polysorbate 80 | 8.0 |
| Propylene glycol | 20.00 |
| Benzyl alcohol | 3 |
| Water | Qs 100 |

### EXAMPLE 2

### Brimonidine 0.15%/Azelaic Acid 10% Solution

| | % by weight relative |
|---|---|
| | to the total weight |
| Ingredients | of the composition |
| Azelaic acid | 10.00 |
| Brimonidine | 0.15 |
| Polysorbate 80 | 2.00 |
| Benzalkonium chloride | 0.05 |
| Ethylene diamine tetra-acetic acid (EDTA) | 0.05 |
| Water | qs 100 |
| 10% sodium hydroxide (Buffer system) | pH 6.3 |

### EXAMPLE 3

### Brimonidine 0.18%/Azelaic Acid 10% Gel

| | % by weight relative to the total weight |
|---|---|
| Ingredients | of the composition |
| Water | Qs 100% |
| Carbopol 980NF | 1.00 |
| Propylene glycol | 5.00 |
| Polyethylene glycol | 5.00 |
| Niacinamide | 2.50 |
| Glycerin | 5.50 |
| Methyl paraben | 0.10 |
| Phenoxyethanol | 0.40 |
| 10% sodium hydroxide | Qs pH 6 +/− 0.5 |
| Azelaic acid | 10.00 |
| Brimonidine | 0.18 |
| Polysorbate 80 | 5.00 |
| Phosphatidylcholine | 5.00 |
| Caprylic/capric | 3.00 |
| triglycerides | |

### EXAMPLE 4

### Brimonidine 0.20%/Azelaic Acid 10% Gel

| | % by weight relative to |
|---|---|
| | the total weight of the |
| Ingredients | composition |
| Water | Qs 100% |
| Carbopol 974PNF | 1.00 |
| Propylene glycol | 5.00 |
| Polyethylene glycol | 5.00 |
| Niacinamide | 2.50 |
| Glycerin | 5.50 |
| Methyl paraben | 0.10 |
| Phenoxyethanol | 0.40 |
| 10% sodium hydroxide | Qs pH 6 +/− 0.5 |
| Azelaic acid | 10.00 |
| Brimonidine | 0.20 |
| Polysorbate 80 | 5.00 |
| Phosphatidylcholine | 5.00 |
| Caprylic/capric | 3.00 |
| triglycerides | |
| Glyceryl stearate | 3.00 |

### EXAMPLE 5

### Brimonidine 0.18%/Azelaic Acid 10% Gel

| | % by weight relative to |
|---|---|
| | the total weight of the |
| Ingredients | composition |
| Water | Qs 100% |
| acrylamide, AMPS | 4.00 |
| copolymer dispersion | |
| 40%/isohexadecane | |
| Caprylic/capric | 3.00 |
| triglycerides | |
| Propylene glycol | 5.00 |
| Polyethylene glycol | 5.00 |
| Niacinamide | 2.50 |
| Glycerin | 5.50 |
| Methyl paraben | 0.10 |
| Phenoxyethanol | 0.40 |
| 10% sodium hydroxide | Qs pH 6 +/− 0.5 |
| Azelaic acid | 10.00 |
| Brimonidine | 0.18 |
| Polysorbate 80 | 5.00 |
| Phosphatidylcholine | 5.00 |

### EXAMPLE 6

### Brimonidine 0.20%/Azelaic Acid 15% Cream

| | % by weight relative to |
|---|---|
| | the total weight of the |
| Ingredients | composition |
| Water | Qs 100% |
| Carbopol 980NF | 0.40 |
| Propylene glycol | 5.00 |
| Polyethylene glycol | 5.00 |
| Niacinamide | 4.00 |
| Glycerin | 5.00 |
| Methyl paraben | 0.20 |
| Propyl paraben | 0.10 |
| Caprylic/capric | 7.00 |
| triglycerides | |
| PEG-6 and PEG 32 | 5.00 |
| stearate | |
| Cetyl alcohol | 3.00 |
| Cetostearyl alcohol | 3.00 |
| Phenoxyethanol | 0.50 |
| 10% sodium hydroxide | Qs pH 6 +/− 0.5 |
| Azelaic acid | 15.00 |
| Brimonidine | 0.20 |
| Polysorbate 80 | 5.00 |
| Phosphatidylcholine | 3.00 |

### EXAMPLE 7

### Brimonidine 0.18%/Azelaic Acid 10% Gel

| | % by weight relative to |
|---|---|
| | the total weight of the |
| Ingredients | composition |
| Water | Qs 100% |
| acrylamide, AMPS | 4.00 |
| copolymer dispersion | |
| 40%/isohexadecane | |
| Propylene glycol | 5.00 |
| Polyethylene glycol | 5.00 |
| Niacinamide | 2.50 |
| Glycerin | 5.50 |
| Methyl paraben | 0.10 |
| Phenoxyethanol | 0.40 |
| 10% sodium hydroxide | Qs pH 6 +/− 0.5 |
| Azelaic acid | 10.00 |
| Brimonidine | 0.18 |
| Polysorbate 80 | 5.00 |
| Phosphatidylcholine | 5.00 |

Process

### EXAMPLE 3 and 4

### Gel

Aqueous Phase:

Solubilize brimonidine in the purified water under stirring using a deflocculator into a container that will serve as receiver for the finish product.

Then, introduce Carbopol 974P NF or Carbopol 980P NF. The mixture is stirred until fully dispersed.

Then, Glycerin, niacinamide, phenoxyethanol and polyethylene glycol introduced under stirring in the aqueous mix.

Preservative Phase:

methyl paraben and propylene glycol are introduced into an additional container. The mix is stirred at a temperature of 45° C. using a magnetic stirrer until methyl paraben is fully solubilized. Add this mix to the aqueous phase.

Neutralization:

The gelling agent neutralizer is introduced up to a pH 6 +/−0.5.

Azelaic Acid Phase:

Azelaic acid, polysorbate 80, caprylic/capric triglycerides, Phosphatidylcholine and optionnally Glyceryl stearate are introduced into an additional container. The mix is stirred using a deflocculator at a temperature of 75° C. until azelaic acid is fully dispersed.

Addition of the Active Phase Azelaic Acid:

At a temperature lower than 50° C., the active phase (azelaic acid) is added to the gel under stirring. The product is homogenized a final time in order to ensure a good dispersion of azelaic acid and the product is then packaged.

### EXAMPLE 5

### Gel

Aqueous Phase:

Solubilize brimonidine in the purified water under stirring using a deflocculator into a container that will serve as receiver for the finish product.

Then, Glycerin, niacinamide, phenoxyethanol and polyethylene glycol are introduced under stirring in the aqueous mix.

Preservative Phase:

Methyl paraben and propylene glycol are introduced into an additional container. The mix is stirred at a temperature of 45° C. using a magnetic stirrer until methyl paraben is fully solubilized. Add this mix to the aqueous phase.

The gelling agent Simulgel 600PHA is added to the aqueous phase under stirring.

Azelaic Acid Phase:

Azelaic acid, Polysorbate 80, caprylic/capric triglycerides and Phosphatidylcholine are introduced into an additional container. The mix is stirred using a deflocculator at a temperature of 75° C. until azelaic acid is fully dispersed.

Addition of the Active Phase Azelaic Acid:

At a temperature lower than 50° C., the active phase (azelaic acid) is added to the gel under stirring. The product is homogenized a final time in order to ensure a good dispersion of azelaic acid and the product is then packaged.

### EXAMPLE 6

### Cream

Aqueous Phase:

Solubilize brimonidine in the purified water under stirring using a deflocculator into a container that will serve as receiver for the finish product.

Then, introduce Carbopol 980 NF. The mixture is stirred until fully dispersed.

Then, Glycerin, niacinamide, phenoxyethanol and Polyethylene glycol introduced under stirring in the aqueous mix.

The mixture is brought to 75° C.

Fatty Phase:

The lipophilic emulsifiers such as PEG-6 and PEG-32 stearate, the oily compounds such as cetyl alcohol, cetostearyl alcohol, Caprylic/capric triglycerides and the preserving agent such as Propyl paraben are introduced under stirring using a magnetic stirrer into an additional container. The mixture is brought to 75° C. until homogeneization.

Emulsification:

The fatty phase is introduced gently into the aqueous phase at a temperature of 75° C. and under stirring using a deflocculator in order to perform the emulsification.

Neutralization:

The gelling agent neutralizer is introduced up to a pH 6 +/−0.5.

Azelaic Acid Phase:

Azelaic acid, Polysorbate 80, Caprylic/capric triglycerides and Phosphatidylcholine are introduced into an additional container. The mix is stirred using a deflocculator at a temperature of 75° C. until azelaic acid is fully dispersed.

Addition of the Active Phase Azelaic Acid:

At a temperature lower than 50° C., the active phase (azelaic acid) is added to the emulsion under stirring. The product is homogenized a final time in order to ensure a good dispersion of azelaic acid and the product is then packaged.

1. Materials and Methods
Macroscopic and Microscopic Observations

Macroscopic observations are carried out at RT (Room temperature), 40° C. and 4° C. in order to check the good appearence of the formulas (no phase separation, homogeneity of the formulation, cosmetic touch . . . ).

Microscopic observations using a AxioScope A1 (under polarized light, objective ×40) are carried out at RT and 4° C. in order to control the Azelaic acid dispersion homogeneity and the good solubity of Brimonidine.
pH Measurement

A mettler Toledo pH-meter is used to measure the pH of the formulas.

Measurement are carried out at RT and 40° C.
Rheological Profile

Measurement of the yield stress of the formulations are carried out with a HAAKE Rheometer (type VT550) at RT.

The yield stress value give us an information about the necessary force to induce a flow.

2. Stability Tests Results

Different physical and chemical testings have been carried out:
Macroscopic and microscopic observations
pH
Rheological profile

The formulations are packed in amber glass jar and stored at room temperature (RT), 40° C. and 4° C.
Physical Stability Results

| Example 3 | | T0 | T1 month | T2 month |
|---|---|---|---|---|
| Macroscopic | RT | Smooth white | Smooth white | Smooth white |
| appearence | | cream gel | cream gel - | cream gel - |
| | | | Conform at | Conform at |
| | | | T0 | T0 |
| | 40° C. | NA | Smooth white | Smooth white |
| | | | cream gel - | cream gel - |
| | | | Conform at | Conform at |
| | | | T0 | T0 |
| | 4° C. | NA | Smooth white | Smooth white |
| | | | cream gel - | cream gel - |
| | | | Conform at | Conform at |
| | | | T0 | T0 |
| Microscopic | RT | Brimonidine is | Conform at | Conform at |
| appearence | | solubilised in | T0. | T0. |
| | | the gel. | | |
| | | Azelaic acid is | | |
| | | dispersed in the | | |
| | | gel. | | |
| | 4° C. | NA | Conform at | Conform at |
| | | | T0. | T0. |
| pH | RT | 5.26 | 5.21 | 5.23 |
| | 40° C. | NA | 4.96 | 4.94 |
| Rheological | RT | 18/37/86 | 18/32/73 | 18/31/73 |
| profile | | | | |
| (4 s⁻¹, | | | | |
| 20 s⁻¹, | | | | |
| 100 s⁻¹) | | | | |

NA: Not applicable

| Example 4 | | T0 | T1 month | T2 month |
|---|---|---|---|---|
| Macroscopic | RT | Smooth white | Smooth white | Smooth white |
| appearence | | cream gel | cream gel - | cream gel - |
| | | | Conform at T0 | Conform at T0 |
| | 40° C. | NA | Smooth white | Smooth white |
| | | | cream gel - | cream gel - |
| | | | Conform at T0 | Conform at T0 |
| | 4° C. | NA | Smooth white | Smooth white |
| | | | cream gel - | cream gel - |
| | | | Conform at T0 | Conform at T0 |
| Microscopic | RT | Brimonidine is | Conform at | Conform at |
| appearence | | solubilised in | T0. | T0. |
| | | the gel. | | |
| | | Azelaic acid is | | |
| | | dispersed in the | | |
| | | gel. | | |
| | 4° C. | NA | Conform at | Conform at |
| | | | T0. | T0. |
| pH | RT | 5.21 | 5.31 | 5.32 |
| | 40° C. | - | 5.00 | 5.00 |
| Rheological | RT | 91/135/227 | 75/117/202 | 71/119/195 |
| profile | | | | |
| (4 s⁻¹, | | | | |
| 20 s⁻¹, | | | | |
| 100 s⁻¹) | | | | |

NA: Not applicable

| Example 5 | | T0 | T1 month | T2 month |
|---|---|---|---|---|
| Macroscopic | RT | Smooth white | Smooth white | Smooth white |
| appearence | | cream gel | cream gel - | cream gel - |
| | | | Conform at T0 | Conform at T0 |
| | 40° C. | NA | Smooth white | Smooth white |
| | | | cream gel - | cream gel - |
| | | | Conform at T0 | Conform at T0 |
| | 4° C. | NA | Smooth white | Smooth white |
| | | | cream gel - | cream gel - |
| | | | Conform at T0 | Conform at T0 |
| Microscopic | RT | Brimonidine is | Conform at T0. | Conform at |
| appearence | | solubilised in | | T0. |
| | | the gel. | | |
| | | Azelaic acid is | | |
| | | dispersed in | | |
| | | the gel. | | |
| | 4° C. | NA | Conform at T0. | Conform at |
| | | | | T0. |
| pH | RT | 4.69 | 4.58 | 4.55 |
| | 40° C. | NA | 4.89 | 4.41 |
| Rheological | RT | 99/194/424 | 137/223/451 | 133/222/430 |
| profile | | | | |
| (4 s⁻¹, | | | | |
| 20 s⁻¹, | | | | |
| 100 s⁻¹) | | | | |

NA: Not applicable

| Example 6 | | T0 | T1 month | T2 month |
|---|---|---|---|---|
| Macroscopic | RT | Smooth white | Smooth white | Smooth white |
| appearence | | cream | cream - | cream - |
| | | | Conform at T0 | Conform at |
| | | | | T0 |
| | 40° C. | NA | Smooth white | Smooth white |
| | | | cream - | cream - |
| | | | Conform at T0 | Conform at |
| | | | | T0 |
| | 4° C. | NA | Smooth white | Smooth white |
| | | | cream - | cream - |
| | | | Conform at T0 | Conform at |
| | | | | T0 |
| Microscopic | RT | Brimonidine is | Conform at | Conform at |
| appearence | | solubilised in | T0. | T0. |
| | | the gel. | | |
| | | Azelaic acid is | | |
| | | dispersed in the | | |
| | | gel. | | |
| | 4° C. | NA | Conform at | Conform at |
| | | | T0. | T0. |
| pH | RT | 5.48 | 5.54 | 5.52 |
| | 40° C. | NA | 5.09 | 5.18 |
| Rheological | RT | 108/168/335 | 95/145/315 | 80/129/313 |
| profile | | | | |
| (4 s⁻¹, | | | | |
| 20 s⁻¹, | | | | |
| 100 s⁻¹) | | | | |

NA: Not applicable

All formulations are physically stable at least 1 month at RT, 40° C. and 4° C.
Chemical Stability Tests

| Example 3 | | | |
|---|---|---|---|
| (% LC Label | Storage | | |
| Claim) | conditions | T0 | T1 month |
| Brimonidine | TA | 103.9% | 101.7% |
| | 40° C. | NA | 102.2% |
| | 4° C. | NA | 100.6% |
| Azelaic acid | TA | 93.8% | 93.7% |
| | 40° C. | NA | 95.9% |
| | 4° C. | NA | 96.7% |

NA: Not applicable

| Example 4 | | | |
|---|---|---|---|
| (% LC Label | Storage | | |
| Claim) | conditions | T0 | T1 month |
| Brimonidine | TA | 103.0% | 101.8% |
| | 40° C. | NA | 102.0% |
| | 4° C. | NA | 101.5% |
| Azelaic acid | TA | 96.9% | 99.8% |
| | 40° C. | NA | 95.7% |
| | 4° C. | NA | 100.1% |

NA: Not applicable

| Example 5 | | | |
|---|---|---|---|
| (% LC Label | Storage | | |
| Claim) | conditions | T0 | T1 month |
| Brimonidine | TA | 98.9% | 100.3% |
| | 40° C. | NA | 99.7% |
| | 4° C. | NA | 100% |
| Azelaic acid | TA | 98.6% | 99.7% |
| | 40° C. | NA | 99.8% |
| | 4° C. | NA | 98.2% |

NA: Not applicable

| Example 6 | | | |
|---|---|---|---|
| (% LC Label | Storage | | |
| Claim) | conditions | T0 | T1 month |
| Brimonidine | TA | 100.8% | 97.3% |
| | 40° C. | NA | NR |
| | 4° C. | NA | 98% |
| Azelaic acid | TA | 94.1% | 98.7% |
| | 40° C. | NA | 95.3% |
| | 4° C. | NA | 98% |

NA: Not applicable
NR: not realized

All formulations are chemically stable at least 1 month at RT, 40° C. and 4° C.

1-27. (canceled)

28. A combination for treating and/or preventing a skin disease, comprising a first compound selected from the group consisting of azelaic acid and salts thereof, and a second compound of the alpha-1 or alpha-2 adrenergic receptor agonist family.

29. The combination according to claim 28, comprising azelaic acid and a compound of the alpha-2 adrenergic receptor agonist family.

30. The combination according to claim 28, wherein the second compound is an alpha-1 adrenergic receptor agonist selected from the group consisting of metaraminol, midodrine, methoxamine, mephentermine, phenylephrine, oxymetazoline, tetrahydrozoline, naphazoline, xylometazoline, and salts thereof.

31. The combination according to claim 28, wherein the second compound is an alpha-2 adrenergic receptor agonist selected from the group consisting of apraclonidine, brimonidine, clonidine, dexmedetomidine, guanbenz acetate, lidamidine, lofexidine, methyldopa, rilmenidine, talipexole, tiamenidine, tizanidine, tolonidine, and salts thereof.

32. The combination according to claim 28, wherein the first compound and the second compound are present in the same composition.

33. The combination according to claim 28, wherein the first compound and the second compound are present separately in separate compositions.

34. The combination according to claim 28, wherein the first compound is present at a concentration of 0.01% to 40% by weight, relative to the total weight of the composition comprising the first compound, and the second compound is present at a concentration of 0.01% to 20% by weight, relative to the total weight of the composition comprising the second compound.

35. A method for treating and/or preventing a skin disease in a subject in need thereof, comprising administering to the subject a combination according to claim 28.

36. The method according to claim 35, wherein the skin disease is rosacea.

37. The method according to claim 36, wherein the rosacea is ocular rosacea.

38. A kit comprising:
(a) a first composition comprising a first compound selected from the group consisting of azelaic acid and salts thereof,
(b) a second composition different from the first composition, the second composition comprising a second compound of the alpha-1 or alpha-2 adrenergic receptor agonist family, and
(c) instructions on using a combination comprising the first composition and the second composition for treating and/or preventing a skin disease, wherein the first and second compositions are applied simultaneously, or separately with a time delay.

39. A method of producing the kit according to claim 38, comprising:
(a) providing to the kit the first composition,
(b) providing to the kit the second composition, and
(c) providing to the kit the instructions.

40. A pharmaceutical or dermatological composition comprising, in a physiologically acceptable medium, at least a first compound selected from the group consisting of azelaic acid and salts thereof and at least a second compound of the alpha-1 or alpha-2 adrenergic receptor agonist family.

41. The pharmaceutical or dermatological composition according to claim 40, comprising, in the physiologically acceptable medium, azelaic acid and a compound of the alpha-2 adrenergic receptor agonist family.

42. The pharmaceutical or dermatological composition according to claim 40, wherein the second compound is an alpha-1 adrenergic receptor agonist selected from the group consisting of metaraminol, midodrine, methoxamine, mephentermine, phenylephrine, oxymetazoline, tetrahydrozoline, naphazoline, xylometazoline, and salts thereof.

43. The pharmaceutical or dermatological composition according to claim 40, wherein the second compound is an alpha-2 adrenergic receptor agonist selected from the group consisting of from apraclonidine, brimonidine, clonidine, dexmedetomidine, guanbenz acetate, lidamidine, lofexidine, methyldopa, rilmenidine, talipexole, tiamenidine, tizanidine, tolonidine, and salts thereof.

44. The pharmaceutical or dermatological composition according to claim 40, wherein the compound of the alpha-2 adrenergic receptor agonist family is brimonidine or a salt thereof.

45. The pharmaceutical or dermatological composition according to claim 40, wherein the first compound represents 1% to 20% by weight, relative to the total weight of the composition.

46. The pharmaceutical or dermatological composition according to claim 40, wherein the concentration of the second compound is 0.01% to 20% by weight, relative to the total weight of the composition.

47. The pharmaceutical or dermatological composition according to claim 40, wherein the concentration of the second compound is 0.02% to 10% by weight, relative to the total weight of the composition.

48. The pharmaceutical or dermatological composition according to claim 40, being for topical application.

49. The pharmaceutical or dermatological composition according to claim 40, being for ocular application.
